(19) United States
(12) Patent Application Publication
(10) Pub. No.: US 2026/0000490 A1
(43) Pub. Date: Jan. 1, 2026

(54) DENTAL/MEDICAL DEVICE INCLUDING POLYDIOXANONE, USE OF SUCH DEVICE FOR GUIDED TISSUE RECONSTRUCTION AND/OR REGENERATION AND PRODUCTION PROCESS OF SUCH DEVICE

(71) Applicant: M3 HEALTH INDÚSTRIA E COMÉRCIO DE PRODUTOS MÉDICOS, ODONTOLÓGICOS E CORRELATOS S.A., Jundiaí (BR)

(72) Inventors: Alberto BLAY, São Paulo (BR); Edvaldo SANTOS DE SOUSA SILVA, Jundiaí (BR); Elaine YOSHIKO MATSUBARA, São Paulo (BR); Jamil AWAD SHIBLI, Guarulhos (BR); Livia PILATTI MENDES DA SILVA, Jundiaí (BR); Sybele SASKA SPECIAN, Santana de Parnaiba (BR); Samy TUNCHEL, São Paulo (BR); Moises COHEN, São Paulo (BR)

(21) Appl. No.: 18/872,425

(22) PCT Filed: Jun. 6, 2023
(86) PCT No.: PCT/BR2023/050185

§ 371 Date: Dec. 6, 2024

Related U.S. Application Data
(60) Provisional application No. 63/349,344, filed on Jun. 6, 2022.

(51) Int. Cl. A61C 8/02; A61F 2/10; A61F 2/14; C08G 63/664; C12N 5/0775

(52) U.S. Cl. CPC A61C 8/0006; A61F 2/105; A61F 2/142; C08G 63/664; C12N 5/0662

(57) ABSTRACT

The present invention relates to a dental/medical device consisting of an absorbable synthetic membrane, preferably white to gray in color, comprising poly(dioxanone) and optionally graphene and stem cells, to be used in guided tissue reconstruction and/or regeneration of bone defects and other connective tissues (gum, skin, cartilage or cornea, for example). Furthermore, the present invention relates to the use of said device for the above-related applications and to the process of preparing said device.

### FIELD OF INVENTION

The present invention relates to a dental/medical device consisting of an absorbable synthetic membrane, preferably white to gray in color, based on poly(dioxanone) (PDO or PDS) and optionally graphene and stem cells, to be used in the tissue reconstruction and/or regeneration of bone defects and other connective tissues such as gingiva, skin, cartilage and cornea, for example, for application in the medical-dental area.

Furthermore, the present invention relates to the use of said device for the above-related applications and to the process of preparing said device.

### BACKGROUND OF THE INVENTION

The principle of guided tissue regeneration (GTR) is based on the use of biocompatible membranes in order to prevent the migration of connective and epithelial tissues to the wound, allowing cells from the periodontal ligament to repopulate the root surface and regenerate the attachment apparatus. tooth. The membrane inhibits the migration of undesirable cells in tissue regeneration. The use and development of biomaterials for tissue regeneration are of great importance, especially for the medical and dental fields.

We highlight below some teachings of the prior art that refer to the present matter:

Guided tissue regeneration in intrabony defects using an experimental bioresorbable polydioxanon (PDS) membrane. A 24-month split-mouth study. Christgau, M. et al. 2002. This prospective split-mouth study compared radiographic and microbiological findings, during the healing process, in deep intraosseous defects after guided tissue regeneration therapy (GTN) with two different bioresorbable membranes. Thirty-one patients with contralateral intraosseous defects were randomly treated with an experimental polydioxanone (PDS) membrane and a polylactic acid (PLA) matrix barrier. Each patient had a pair of contralaterally located deep intraosseous defects, interproximal periodontal defects with a probing pocket depth (PPD) of at least 6 mm, and radiographic evidence of angular bone loss of at least 4 mm at baseline. After 6, 12, and 24 months, the healing results were evaluated through clinical examinations: gingival recession (REC), probing pocket depth (PPD), clinical attachment level (CAL), relative vertical attachment gain (V-rAG), quantitative digital radiographic subtraction (amount and area of bone density changes) and microbiological analysis. Postoperative membrane exposures occurred at 14 PDS treated sites and 2 PLA treated sites. At 6, 12, and 24 months after surgery, both membranes provided a significant gain in CAL [median values: 6 months (PDS vs. PLA: 3.0 vs. 3.0 mm); 12 and 24 months (PDS vs. PLA: 4.0 vs. 4.0 mm)], which corresponded to a V-rAG of 57.1% (PDS) vs. 62.5% (PLA) after 24 months. Sites treated with PDS and PLA showed significant bone density gain 6, 12, and 24 months after surgery. Bone density gain after 12 and 24 months, in radiographic analyses, was statistically significantly greater in PDS than in PLA sites. After 24 months of healing, 38.8% of the initial defect area at PDS sites and 41.8% of the initial defect area at PLA sites showed bone density gain, but these differences were not statistically significant. Microbiological culture revealed similar bacterial loads at the PDS and PLA sites during the first 12 months. In conclusion, this 24-month study indicates that PDS and PLA membranes can provide similar favorable regeneration results in deep intraosseous periodontal defects, although considerably more postoperative membrane exposures should be expected at PDS-treated sites.

Regenerative periodontal surgery in interproximal intrabony defects with biodegradable barriers. Darter CE. et al. 2000. The effects of guided tissue regeneration (GTN) using 2 different degradable barriers (polylactide acetyl tributyl citrate; polydioxanone-PDS) on intraosseous defects were compared. Fifteen patients provided 15 pairs of similar contralateral periodontal defects. Each defect was randomly assigned to treatment with the devices: polylactide acetyl tributyl citrate (control [c]—Guidor Matrix Barrier, Guidor AB, Huddinge, Sweden) or PDS (test [t]—Mempol, Ethicon GmbH & Co. KG, Norderstedt, Germany). In the healing phase, one patient on one side (PDS) developed an infection. After 6 months of surgery, clinical measurements: plaque index (PII), gingival index (GI), probing pocket depth (PPD) and vertical probing insertion levels (PAL-V) were performed. The results showed that barrier exposure was commonly observed in both groups (control/test): 5/4 after 7 days, 9/11 after 14 days, and 11/12 after 28 days post-surgical. Four weeks after surgery, 77% of all barriers were exposed to some degree. However, both treatments revealed a significant reduction in GI (p<0.05), reduction in PPD [−4.63+1.85 mm (t), −4.17+1.89 mm (c); p<0.001] and PAL-V gain [3.97+1.17 mm (t), 3.40 mm+1.40 (c); p<0.001] 6 months after surgery. Regarding the reduction of GI and PPD, as well as the gain of PAL-V, there were no statistically significant or clinically relevant differences between the test and the control.

Guided tissue regeneration with bioabsorbable barriers: intrabony defects and class II furcations. Eickholz, P. et al. 2000. The authors compared the effects of guided tissue regeneration (GTR) using 2 different bioabsorbable barriers (control: polylactide acetyl tributyl citrate; test: polydioxanone) in 21 patients with 22 pairs of similar contralateral defects (30 intraosseous and 14 Class II furcation lesions), each defect was randomly assigned to treatment with control (c) devices (Guidor Matrix Barrier) or test (t) (Mempol, Ethicon). At baseline and 12 months after surgery, clinical measurements such as: plaque index (PI), gingival index (GI), probing depth (PD), and vertical and horizontal clinical attachment loss (CAL-V; CAL-H) and standardized radiographs were obtained. The results revealed that barrier exposure was commonly observed in both groups. Four weeks after surgery, 61% of all barriers were exposed to some degree. However, both treatments revealed a significant GI reduction (P<0.005), PD reduction (−3.08+/−2.29 mm [t]; −3.52+/−2.67 mm [c]; P<0.001) and CAL-V gain (2.44+/−2.29 mm [t], 2.80 mm+/−2.21 [c]; P<0.001) at 12 months after surgery in all defects. Within intraosseous defects, bone filling was significant (2.03+/−1.70 mm [t]; 1.91+/−1.20 mm [c]; P=0.001), and in lesions of furca, there was also a significant, albeit small, CAL-H gain (0.79+/−0.68 mm [t]; 1.13+/−1.44 mm [c]; P<0.05). No statistically significant or clinically relevant differences between test and control at 12 months after surgery were observed for the analyzed data.

Comparison of two types of synthetic biodegradable barriers for GTR in interproximal infrabony defects: clinical and radiographic 24-month results. Kim, T. S. et al. 2003. This study compared the effectiveness of guided tissue regeneration (RTG) using polylactide acetyl tributyl citrate and polydioxanone (PDS) in three- and two-walled infrabony defects. Fifteen patients provided 15 pairs of similar contralateral periodontal defects: 12 predominantly two-walled and 18 predominantly three-walled infrabony defects. Each defect was randomly assigned to treatment with polylactide acetyl tributyl citrate (control-Guidor matrix barrier) or polydioxianone devices (test-Mempol). At baseline, 6, 12, 18, and 24 months after surgery, clinical measurements were taken, and standardized radiographs obtained (not at 18 months). Both treatments revealed a significant reduction in gingival index, reduction in probing depth, and gain in insertion level of vertical probing 24 months after surgery. Both treatments showed slight resorption of the alveolar bone crest after 24 months, which did not reach statistical significance. A statistically significant bone gain in the infrabony pockets was measured for both treatment options at 24 months. Regarding the gingival index and the reduction in probing depth, as well as the vertical probing insertion level and bone gain, there were not statistically significant or clinically relevant differences between the test and control barriers. The use of both biodegradable barriers in GTR therapy can be recommended.

Guided tissue regeneration with bioabsorbable barriers. II. Long-term results in infrabony defects. Eickholz, P. et al. 2004. The aim of this 5-year randomized controlled clinical trial was to evaluate the long-term outcomes after guided tissue regeneration (GTN) therapy of infrabony defects using two absorbable barriers. The study included 15 patients with a pair of contralateral infrabony defects and moderate to severe periodontitis. Each patient received a polydioxanone (test: T) and an acetyl tributyl citrate polylactide barrier (control: C) by random assignment. At baseline, 12 and 60+/−3 months after surgery, clinical parameters and standardized radiographs were obtained. Vertical bone levels (PBL-V) were measured during surgery and 60+/−3 months later by transgingival bone probing. Thirteen patients were available for the 60-month examinations. Twelve and 60+/−3 months after GTR, the vertical insertion gain (CAL-V) found was statistically significant (P≤0.001) for both groups (T12: 3.5+/−1.5 mm; T60: 2.2+/−1.8 mm; C12: 4.0+/−0.9 mm; C60: 2.4+/−1.0 mm). However, from 12 to 60 months after therapy, both groups experienced significant CAL-V loss (P<0.05): two defects in the test group and three in the control group had CAL-V loss>2 mm compared to the 12-month review. Twelve and 60+/−3 months after surgery, radiographically measured bone fill was statistically significant (P<0.05) for both groups (T12: 1.2+/−1.3 mm; T60: 1.5+/−2.2 mm; C12: 0.9+/−1.4 mm; C60: 1.0+/−1.6 mm). Furthermore, 60 months after surgery, the PBL-V gain found in both groups (test: 1.8+/−2.3 mm; control: 2.2+/−1.8 mm) was significant (P<0.05). No statistically significant differences were found between the test and the control regarding the gain of CAL-V and PBL-V at 60 months after surgery. In conclusion, the CAL-V gain achieved after GTR therapy in infrabony defects using both absorbable barriers were stable after 5 years in 21 of 26 defects (81%).

Guided tissue regeneration with bioabsorbable barriers III 10-year results in infrabony defects. Pretzl, B. et al. 2009. This article evaluated 10-year outcomes after LTR therapy of infrabony defects using two bioabsorbable barriers in a randomized controlled trial. In 15 patients with periodontitis, 15 pairs of infrabony defects were treated. For each patient, one defect received a polydioxanone (test: T) and the other received a barrier of acetyl tributyl citrate polylactide (control: C) by random assignment. At baseline, 12 and 120+/−6 months after surgery, clinical parameters and standardized radiographs were obtained. For the 120-month exams, only 9 patients, out of the initial 15, attended. Twelve and 120+/−6 months after therapy the gain in vertical probe insertion level (PAL-V) found in both groups (T12: 3.9+/−1.6 mm; T120: 2.4+/−1.8 mm; C12: 4.0+/−1.1 mm; C120: 2.4+/−1.7 mm), was statistically significant (p≤0.004). From 12 to 120 months, both groups experienced PAL-V loss (T: 1.4+/−1.5 mm, p=0.021; C: 1.6+/−2.5 mm, p=0.09). After 120 months, two teeth were lost in the control group (one for a periapical lesion and another for an unknown reason). The study showed no statistically significant differences between the two groups regarding PAL-V gain at 120 months after surgery.

Document US 2020/0330641 discloses a suture membrane composed of graphene oxide (due to its biocompatibility, bacteriostatic action and hydrophilicity) and polymers. It makes use of electrospinning technique in the production of polymeric fibers. Furthermore, it provides for the surgical use of the fibrous biocomposite membrane, biodegradable, made of graphene oxide, as a medical dressing, surgical sutures or drug carrier.

The document KR 101636778 discloses a method for producing a biodegradable nanofiber sheet for tissue regeneration. It makes use of graphene oxide, given its properties, particularly hydrophilicity and flexibility, which play an important role in cell proliferation and differentiation. It uses the electrospinning technique to generate graphene oxide fibers and synthetic and natural polymers, as a support for tissue regeneration. Among these polymers is polydioxanone.

Document CN 105126167 describes bone tissue repair and reconstruction, and particularly to a method of preparation and support for a 3D printed, porous titanium-based alloy surface functionalized biological coating for bone tissue repair and reconstruction. The functionalized porous frame made of 3D printed metal features a functionalized bioactive nanocomposite coating on the surface of fibroin oxide-graphene silk. It also employs polymers such as polydioxanone.

Therefore, in the prior art, there is no solution equivalent to the one presented here in the present invention that combines technical differences, economic advantages, safety and reliability.

### OBJECTIVES OF THE INVENTION

Thus, it is an objective of the present invention to provide a device to be used in the reconstruction and/or guided tissue regeneration of bone defects and other connective tissues such as gingiva, skin, cartilage and cornea, for example.

It is another objective of the present invention to provide a single-use dental/medical device, supplied in a sterile condition and available in various models and dimensions.

It is another objective of the present invention to provide a solution indicated for patients who need guided tissue reconstruction and/or regeneration of bone defects and other connective tissues such as gingiva, skin, cartilage and cornea, for example.

Furthermore, it is an objective of the present invention to provide a solution that is reabsorbed by the body gradually and replaced by newly formed bone tissue and/or soft tissue (gingiva, skin, cartilage or cornea), during the process of repair or guided tissue regeneration.

It is an objective of the present invention to provide a solution which has sub and micrometric fibers, mechanical and biological barrier with morphology and surface topography that mimics the extracellular matrix.

It is the objective of the present invention to provide a solution with physicomechanical and biological barrier functionalities that can be used in different types of surgeries and applications in the orthopedic medical field, such as dura mater reconstruction, periosteum replacement, yellow ligament replacement (spine), mechanical barrier for graft maintenance, biological barrier in grafting techniques, trauma, bone tissue reconstruction, bone tissue coverage after osteophyte removal and reconstruction of defects or cranial closure.

It is the objective of the present invention to provide a product presenting functional and mechanical characteristics satisfactory to the intended use.

It is the objective of the present invention to provide a product that mimics an extracellular matrix of connective tissue, providing mechanical strength for efficient handling, as well as high absorption of fluids and molecules.

### BRIEF DESCRIPTION OF THE DRAWINGS

The present invention will be described on the basis of the drawings attached hereto, which illustrate:

FIG. 1 illustrates the structure of the present invention;

FIG. 2 illustrates the structure of the present invention in a preferred embodiment (comprises graphene);

FIG. 3 illustrates the structure of the present invention in a preferred embodiment in which (A) the present invention comprising 0.52% reduced graphene oxide, (B) the present invention comprising 0.71% reduced graphene oxide and (C) the present invention comprising 1.35% reduced graphene oxide;

FIG. 4 is a graph that shows the relationship between the extensional stress and the deformation of the present invention in the configurations illustrated in FIG. 3;

FIG. 5 illustrates chondrocytes integration-chondrogenic differentiation on the present invention at incubation days 5 (a and b) and 11 (c and d).

### SUMMARY OF THE INVENTION

The present invention achieves these and other objectives by means of a dental/medical device particularly indicated for guided tissue reconstruction and/or regeneration of bone defects and other connective tissues consisting of an absorbable synthetic membrane comprising poly(dioxanone) with a substantially rectangular cross section with thickness varying between 0.05 mm and 2.00 mm, width varying between 15 mm and 200 mm and length varying between 20 mm and 200 mm.

The present invention achieves these and other objectives by the use of the above device to be for guided tissue reconstruction and/or regeneration of bone defects and other connective tissues such as gingiva, skin, cartilage and cornea, for example.

Furthermore, the present invention achieves these and other objectives by means of a process for preparing the device of the present invention which comprises the following steps:
a. raw material is placed in a vacuum stove at room temperature;
b. the raw material is weighed to prepare the dissolution with organic solvents;
c. employee performs the machine setup by inserting the appropriate program for the operation;
d. after loading the machine with the polymeric dissolution, the machine is activated, and the production of the membrane takes place with a predetermined flow rate;
e. the membrane blanket is removed and placed in a vacuum stove for residual organic solvent removal;
f. the blanket is sent to the CO2 laser cutter for the membrane cutting process;
g. membranes are stored in an airtight container;
h. the membranes are individually packed.

### DETAILED DESCRIPTION OF THE INVENTION

The present invention, in a first preferred embodiment, relates to a dental and/or medical device comprising a polydioxanone membrane. Furthermore, said membrane has sub and micrometric fibers whose physicochemical and morphological characteristics facilitate the diffusion of biological fluids and cell adhesion.

Thus, the device of the present invention is indicated to be used in the reconstruction and/or guided tissue regeneration of bone defects and other connective tissues such as gingiva, skin, cartilage or cornea, for example.

The device of the present invention comprises a membrane prepared from poly(dioxanone) as it is a resorbable alloplastic material. This type of material is indicated for bone repair because it is concomitantly replaced by neoformed tissue. In this way, the device of the present invention can be used both as a mechanical barrier to maintain the defect space, and as a mesh for the volume increase/reconstruction of bone tissue and soft tissues. The physical structure and composition of the membrane allows for controlled resorption within an estimated period of 3 to 12 months.

Therefore, said membrane is synthetic resorbable poly(dioxanone) and has a microstructure in the form of a mesh that works as a mechanical barrier promoting space maintenance and an efficient tissue repair or regeneration process.

The decision to use an absorbable polymer is due to the fact that heterogenous natural polymers are generally difficult to handle, have physicochemical characteristics that are difficult to control in each batch, have a degradation rate that is difficult to modify, and their purification and of sterilization are not simple. In addition, they present a greater risk of cross-contamination from the source of the material (pathogens and viruses) and a greater possibility of immunoreactivity due to the presence of proteins produced in other species. Furthermore, the induction of severe local inflammatory reaction after the use of collagen membranes has already been reported.

The device of the present invention is for single use, supplied in a sterile condition and available in various designs and dimensions.

Preferably, the device of the present invention is presented in the following dimensions: thickness ranging from 0.05 mm to 2.00 mm, width ranging from 15 mm to 200 mm and length ranging from 20 mm to 200 mm.

Preferably, the present invention is formed by filaments with diameters between 500 nanometers to 2 micrometers.

Further, the present invention comprises a technical differential since it is produced by electrospinning which confers higher resistance in relation to the PDS plate process which is molding/injection as shown in the table below:

| | | Tensile | Eloongation |
|---|---|---|---|
| Medical | Raw | Strength | at |
| device | material | (MPa) | break(%) |
| present | PDQ | 3.93 ± 0.18 | 280.3 ± 26.4 |
| invention | | | |
| Bio Gide | Collagen | 4.8 | 46.8 |
| | | 1.79 | |
| BioMend | Collagen | ~4.5 | X |
| Jason | Collagen | 13 | 17.9 |
| PDS | PDQ | 2.57 | X |
| | | 2.31 | |

In a second preferred embodiment, the present invention comprises graphene or other compounds made from carbon such as nanocarbon and carbon tubes. The presence of these components adds mechanical strength to the device of the present invention and confers antibacterial property.

In a third preferred embodiment, the present invention comprises stem cells preferably being autogenous adult mesenchymal stem cells obtained from dermal punch of the patient.

In a fourth preferred embodiment of the invention, the present invention comprises graphene and stem cells. This embodiment is illustrated in FIG. 5: on human adipose-derived stem cells (hASCs) chondrogenic differentiation fiber coating and morphological change were noted after 11 days of differentiation, on both evaluated membranes, with a great structural organization on the present invention.

Embodiments of the present invention that comprise stem cells have the following characteristics:
customized product;
use of adult mesenchymal stem cells from adipose tissue: obtained from dermal punch: easily accessible source/immunomodulatory action/accelerates regenerative processes.
flexibility in use: dental and/or orthopedic;
by the use of stem cells, it is possible to use the device of the present invention not only for cartilage regeneration, but also for the regeneration of other tissues, such as bone tissue.

### Application of the Device of the Present Invention

The application of the device must be done in an outpatient or hospital environment.

The techniques of use and application of the device of the present invention may vary according to the preference of the surgeon (doctor or dentist), being up to him to choose the therapeutic approach, model and dimensions of the product, application technique, the use of a fixation or use of complementary biomaterials, as well as the criteria for monitoring and evaluating the results of the surgery.

Proper site preparation (recipient bed) involves treating the entire area that will be covered by the device. The cavity and/or bone tissue or soft tissue and the grafted area must have a homogeneous surface and the final shape of the desired recipient bed already prepared, as well as controlled bleeding, so that the device has a perfect adaptation to the recipient bed and achieve the expected results.

In the manipulation and implantation procedure of the device of the present invention, the surgeon should use only appropriate and sterile surgical instruments. And, before the surgical procedure, the surgeon (doctor or dentist) must carry out a rigorous preoperative planning, through clinical and imaging exams (radiographs or computed tomography). It should also consider the need for a detailed anamnesis and complementary tests on the patient's general health (complete blood count, coagulogram, calcium dosage, etc.), since this information directly influences the biological response of the organism to the installation of the device of the present invention and then define a surgical plan in advance, with the selection of the model and size of the device best suited to the condition of your patient.

The surgical protocol must be performed according to the surgeon's references and previous experiences, always considering the most appropriate choice of the model and size of the device, the technique, the installation sequence and the use of fixation methods (sutures, tacks or screws), always based on in conventional and established therapeutic techniques of guided tissue regeneration and bone grafting.

The present invention presents numerous technical and economic advantages when compared to the prior art, some of which are listed below:
the device of the present invention guarantees a high swelling efficiency;
the device of the present invention acts as a mechanical barrier promoting space maintenance and efficient guided tissue regeneration;
the device of the present invention has a morphology and surface topography similar to the extracellular matrix, whose physicochemical and morphological characteristics facilitate the diffusion of biological fluids and cell adhesion;
the device of the present invention is designed and manufactured ensuring safety and efficacy in relation to systemic toxicity;
the device of the present invention is safe and effective in terms of biological compatibility. Physicochemical analyzes and pre-clinical tests ensure its characteristics, performance and functionality;
it is an absorbable product and has no risks deriving from substances released from it, according to the test for the identification of trace elements and gel permeation chromatography;
the device of the present invention was designed and manufactured in order to eliminate the risks linked to temperature conditions, ensuring the integrity of the product while it is in its validity period;
synthetic membranes reduce the risk of cross-contamination and immunoreactivity;
the present invention is a guided tissue regeneration device indicated to intraosseous defects due to congenital, post-traumatic, post-surgical problems, or as a result of diseases, such as periodontal and peri-implant lesions;
   the present invention promotes alveolar ridge augmentation/reconstruction associated with bone grafts or synthetic bone substitutes for guided tissue regeneration;

The present invention acts as a barrier favoring the maintenance of the space of the bone cavity or the space created for the increase of alveolar rim.

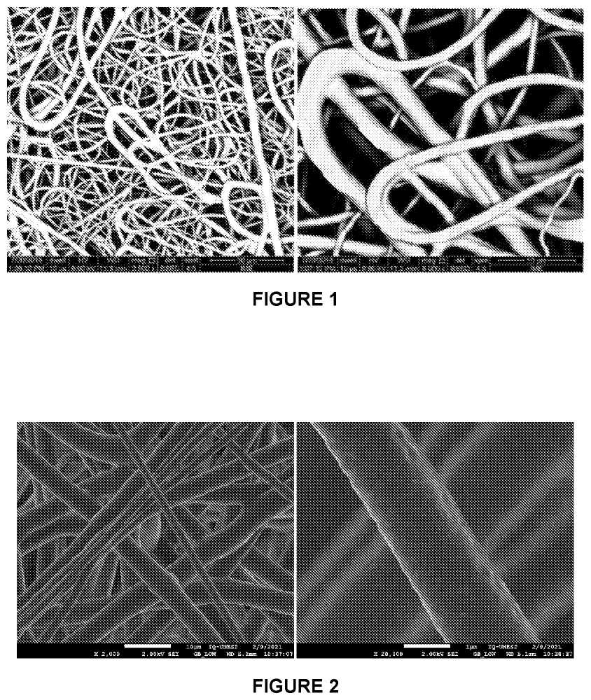

In a preferred embodiment of the present invention, the dental/medical device particularly indicated for guided tissue reconstruction and/or regeneration of bone defects and other connective tissues including gums, skin, cartilage or cornea comprises graphene (reduced graphene oxide). The structural difference of this preferred embodiment can be observed from FIG. 1 compared to FIG. 2. Preferably, the said device comprises graphene, preferably reduced graphene oxide in a concentration ranging between 0.52% and 1.35%.

| | Average Fiber | Extensional | Extensional |
|---|---|---|---|
| | Diameter | Tension | Deformation |
| | (μm) | (MPa) | (%) |
| Membrane | 2.20 μm | 0.05 | 0.07 |
| rGO 0% | | | |
| Membrane | 2.87 μm | 0.0036 | 0.60 |
| rGO 0.52% | | | |
| Membrane | 3.41 μm | 0.0035 | 0.65 |
| rGO 0.71% | | | |
| Membrane | 3.27 μm | 0.005 | 1.98 |
| rGO 1.35% | | | |

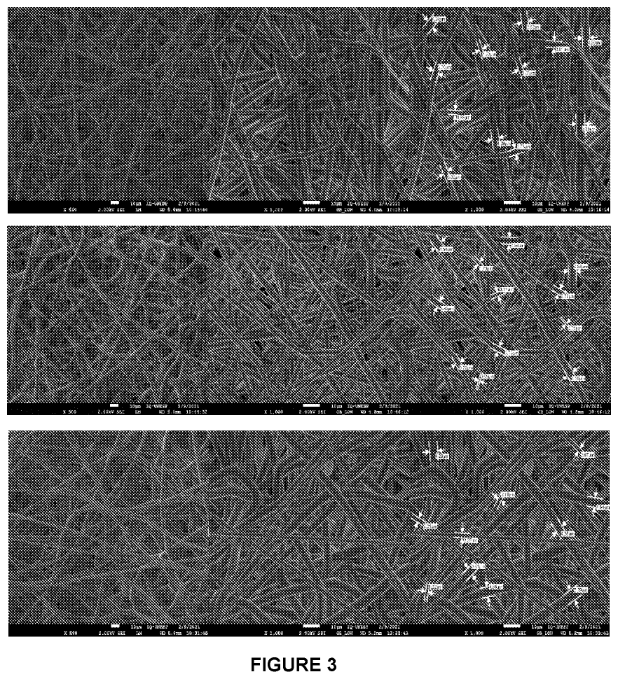

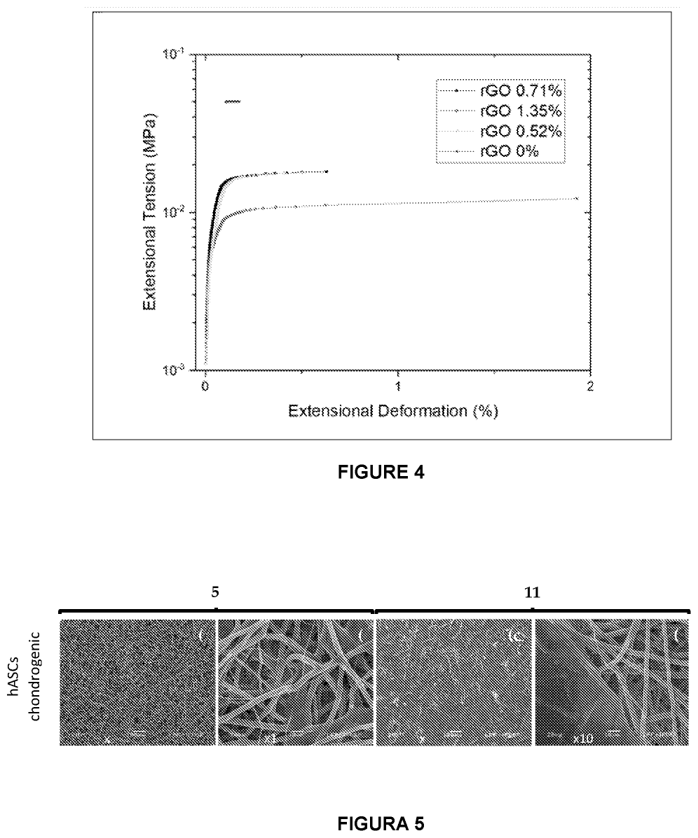

Still, it is possible to observe the structural difference of these concentrations in FIG. 3 in which (A) illustrates the present invention comprising 0.52% reduced graphene oxide, (B) illustrates the present invention comprising 0.71% reduced graphene oxide and (C) illustrates the present invention comprising 1.35% reduced graphene oxide.

As a result obtained with the addition of reduced graphene oxide to the present invention, The control membrane presents the highest value of extensional stress and the lowest deformation, favoring rupture.

### Tests

### 1. Physical and Mechanical Characterization

Mechanical tests were performed based on ASTM D882: 2012-Standard Test Method for Tensile Properties of Thin Plastic Sheeting. The specimens were produced in the following dimension: 50 mm×10 mm, in the longitudinal direction of the mesh. The specimens, n=5 from each batch, sterile by ethylene oxide (EtO) or non-sterile, were tested in an Instron model 5569 universal testing machine, with a 500N load cell, at a speed of 50 mm·min-1, at 23QC and 50% R.H. The results obtained revealed that sterilization by EtO did not change the mechanical properties of the membrane, proving to be satisfactory for this product. Additionally, the results found in the literature in relation to collagen membranes show that the device of the present invention presented tensile strength and tensile strength similar or superior to the tests performed with Bio-Gide (Geistlich), Jason (Botiss), BioMend (Zimmer) (1) and strain at break superior to all collagen membranes in the national and international market (1-3). Regarding the Johnson & Johnson PDS membrane, the mechanical properties of the device of the present invention were shown to be superior compared to the literature data (2).

### 2. Chemical/Material Characterization

### Trace Elements

The methodology used for the tests and the reference parameters were based on the ISO10993-1, ISO10993-17, ISO10993-18 and ABNT NBR ISO 13175-3:2013 standards, commonly used for the characterization of calcium phosphates for medical application, considering that the total amount of heavy metals in the product is the sum of the following elements: lead, mercury, bismuth, arsenic, antimony, tin, cadmium, silver, copper and molybdenum. The results showed that for all analyzed elements the values are below the quantification limit (LO) of the equipment. The batches showed acceptable levels of trace elements in accordance with those defined by ABNT NBR ISO 13175-3:2013.

### 3. Swelling Rate According to ASTM 570

To determine the swelling rate, a sample of 5 was established for each thickness of the device of the present invention: 0.25 mm, 0.5 mm and 1.0 mm. The values obtained in the experiment showed a mean percentage rate of swelling for the 0.25 and 0.5 mm membranes of 436.55±22.62% and 425.23±14.99%, respectively, and for the device with thickness of 1 mm of 239.46±11.54%, in relation to the dry mass of each type of membrane after 24 h immersed.

The results disclosed that, for the thinner membranes (0.25 and 0.5 mm), the maximum swelling rate occurred in the first minute of immersion and for the 1 mm thick membrane, the swelling stabilized after 2 h, having an average swelling rate of 400% in relation to its dry weight for thicknesses of 0.5 and 0.25 mm and up to 200% for thicknesses of 1 mm.

### 4. Scanning Electron Microscopy

The SEM images show a morphological structure with a random weave of fibers in submicron order. The laser cutting proved to be effective in the images obtained by SEM, whose images revealed that the difference in the morphology of the membrane in the region distant and close to the laser cutting site is restricted to a width of the order of 30 μm.

### 5. DSC (Differential Exploratory Calorimetry)

Differential Scanning calorimetry (DSC-Differential) scanning calorimetry), was performed by analyzing the DSC curves of the sample without sterilization, using a capped aluminum sample holder. The sample was subjected to the following temperature program: Heating from −10° C. to 120° C. at 10° C./min; 5 min isotherm at 120° C.; Cooling from 120° C. to −10° C. at 10° C./min; 5 min isotherm at −10° C.; Heating from −10° C. to 350° C. at 10° C./min. The cycle presented was carried out under a dynamic atmosphere of nitrogen gas (N2) at a flow rate of 50 ml/min. The experiment was carried out according to ASTM D3418: 2015, using a Netzsch model DSC 214 Polyma equipment. The result showed that the sample of the device of the present invention without sterilization showed satisfactory results regarding the thermal property. The data obtained for melting temperature and crystallization temperature for the product corroborate the literature data for the PDO polymer (4,5).

### 6. Product Integrity

According to the results presented in the reports AFK01834/20, AFK0476/21 and AFK0653/21, the samples of the device of the present invention at all times tested obtained satisfactory percentage of crystallinity (% Xe) results, similar to the literature data. The values obtained for degree of crystallinity (mean and standard deviation, T1=49±1.4% Xe; T2=44±0.8% Xe; T3=50±0.94% Xe) have acceptable values and are within the determined in design (40-60% Xe) for the intended use in a way that does not compromise its effectiveness (provided that it is kept under suitable temperature conditions). The samples produced showed stability and repeatability among their respective triplicates for up to 12 months.

### 7. Maximized Dermal Sensitization

The Maximized Dermal Sensitization assay consists of analyzing the material's ability to cause an immunologically mediated skin reaction to a substance, characterized by the appearance of edema and/or erythema. The maximized method uses an adjuvant capable of stimulating the immune response in order to enhance the sensitivity of the method (Freund's Complete Adjuvant-FCA). As it is a solid material, the device of the present invention had to undergo an extraction process to be inoculated (see ISO 10993-12: Sample preparation and reference materials, 2012). For this, the device was extracted at 37QC for 72 hours in a Shaker incubator at 100 rpm in a proportion of 6 cm² of the membrane, for 1 ml of 0.9% sodium chloride solution. The liquid resulting from this process (extract) presented a homogeneous and colorless appearance. After extraction, tests were performed with guinea pigs (Gavia porcellus-Dunkin Hartley), in which intradermal injections were applied with control and extracts of the test substance in different dilutions, in addition to the cutaneous exposure of these products. The results showed that no dermal reactions were observed in the experimental and control groups, and the test was terminated. Thus, according to the methodology applied according to ISO 10993-10: Biological evaluation of medical devices-Tests for irritation and skin sensitization, the test substance was found to be non-sensitizing.

### 8. Intracutaneous Reactivity

Intracutaneous Reactivity test consists of the evaluation of local adverse effects occurring after the inoculation of a substance intracutaneously in a single dose. As it is a solid material, the device of the present invention had to undergo an extraction process to be inoculated (see ISO 10993-12: Sample preparation and reference materials, 2012). The test was performed on rabbits (Oryctolagus cuniculus-New Zealand), randomly selected and shaved in the dorsum region, in an area of approximately 10×15 cm, approximately 24 hours before the application of the test substance. After shaving, the back of the animals was divided into a test area and a control area with the aid of a hydrographic pen and the device extract was applied to 5 sites in the test area, in a volume of 0.2 ml per site, intracutaneously, totaling 1 ml of test substance per animal. The extraction vehicle (sodium chloride 0.9%) was applied in the control area under the same conditions of application of the device. Application sites were evaluated 24, 48 and 72 hours after application and were classified as to the absence or presence of edema and erythema. The result showed that none of the animals presented edema or erythema neither for the sites (Test) nor for the control sites (sodium chloride 0.9%). Thus, according to the methodology applied according to ISO 10993-10 Biological evaluation of medal devices-Tests for irritation and skin sensitization, the test substance was found to be satisfactory.

### 9. Acute Systemic Toxicity

Acute systemic toxicity is the assessment of possible health risk and adverse effects caused by a single exposure to a substance. These studies provide information on systemic toxic effects and serve as a basis for estimating the safety of the substance. The tests carried out by were based on the ISO 10993-11: Biological evaluation of medical devices-part 11: Tests for systemic toxicity (2006) and its 2017 update. As it is a solid material, the test substance had to undergo an extraction process in 0.9% sodium chloride solution to be inoculated, according to ISO 10993-12: Sample preparation and reference materials, 2012. The test substance extract was applied intravenously in a volume of 50 ml/kg of animal weight; and the extraction vehicle was applied to the control group, under the same conditions as the test substance in 10 mice (Mus musculus-Swiss). After application, the animals were regularly observed at 1, 24, 48 and 72 hours after application, for the presence of toxic signs, such as changes in the skin, eyes, respiratory, cardiovascular and gastrointestinal systems, changes in motor activity, salivation, seizures, hairy erection, weight loss and death. The test substance was evaluated according to pharmacopoeia criteria, which determine that if all animals survive and do not show clinical signs of toxicity, the test substance will be considered in compliance with the adopted requirements. After the test, no signs of systemic toxicity or death were observed in the test group, thus, there was no need for gross necropsy to assess toxicity. Therefore, according to the methodology applied according to ISO 10993-11: Biological evaluation of medical devices-Tests for systemic toxicity, the test substance was found to be satisfactory.

### 10. Subchronic Systemic Toxicity

The tests performed were based on the ISO 10993-6: Biological evaluation of medical devices-part 6: Tests for local effects after implantation (2007) and in the ISO 10993-11: Biological evaluation of medical devices-part 11: Tests for systemic toxicity (2006) and update (2016). The subchronic systemic toxicity test assesses the existence of possible health risks and/or possible adverse effects caused by repeated and/or continuous exposure to a substance. The implantation assay evaluates its local effects after direct contact of the test substance with the tissues to which it is exposed. Together these assays provide information on possible systemic and target organ toxic effects, characterize the history and evolution of tissue response and serve as a basis for estimating the biological safety of the test substance. In this sense, the present trial aimed to evaluate the local and systemic effects of the device product of the present invention applied on bone tissue (according to the indication and purpose for which the product is intended) for 90 days. The tests were carried out in rabbits (Oryctolagus cuniculus-New Zealand), which underwent insertion of the device in the tibia, under a specific protocol. After the procedure, the animals were weighed and evaluated for clinical signs of toxicity that included, but were not limited to, convulsion, mutilation, prostration, ataxia, tremors, local inflammation, dyspnea, tearing, salivation, diarrhea, piloerection, and cachexia, at baseline and weekly throughout the study period. After 90 days, blood samples were collected from the animals and they were submitted to necropsy and collection of the following organs: liver, spleen, left kidney, left adrenal, testis/ovary, proximal (popliteal) and distal (mesenteric) lymph nodes and sites of implantation. After the analyses, the results indicated the absence of local and systemic toxic effects in rabbits, which was sufficient to demonstrate its safety, efficacy and potential performance for its intended use. The results presented are also sufficient to justify the absence of the need to carry out the tests: Hemocompatibility (ISO10993-4) and immunotoxicological (ISO10993-20); given the absence of blood and immunological alterations; Chronic toxicity (ISO10993-11), given the absence of systemic changes that would indicate the need for a longer follow-up period; Reproductive and developmental toxicity (ISO10993-3), given the absence of histopathological changes in Organs reproductive organs; Toxicokinetics (ISO10993-16), given the absence of toxic potential demonstrated by the device of the present invention.

### 11. Tests in Animal Models-Implementation 180 Days

The bone implant test evaluates the local effects after the implantation of a material in an animal species, with the objective of characterizing the history and evolution of the tissue response after the implantation of a medical device, also evaluating its biological safety. Both the device of the present invention (test) and the reference product (control) were inserted over rounded incisions of 3 mm in diameter in the region close to the tuberosity of the tibia of Rabbits-Oryctolagus cuniculus-New Zealand. The bone tissues of the animals, in the test sites, presented a focally extensive area composed of a discreet to moderate layer of newly formed bone tissue, with greater cellularity of osteocytes, discreet to moderate amount of osteoblast rhymes, and fibers in undefined orientations, in addition to a slight to moderate amount of non-mineralized bone matrix, separated by a cementing line, from the lamellar region with haversian systems, where a slight to moderate alteration of tissue architecture can be observed. Minimal focal capillary proliferation (1-3 buds) associated with bone formation was observed. In the semi-quantitative assessment of local bone reaction, the mean of the test sites was scored at 13.6, while that of the control sites was 14.6, resulting in a tissue reaction index of −1.0 (non-reactive compared to control). The animals did not show significant alterations during the experimental period, nor significant macroscopic alterations in the necropsy exam, indicating absence of toxicity for the presented parameters.

### 12. Testing on Sheep

### 12.1 Study Summary

The present study aimed to analyze the safety and efficacy of cartilaginous repair capacity and action on the joint inflammatory response in sheep of the present invention Test Item. The methodology consisted of the evaluation of implantation sites after the treatment of chondral lesion in adult female sheep (Ovis aries), by means of anatomopathological analyses when compared to the control or reference treatments.

The chondral lesion of 10 mm in diameter was induced in the support center of the medial condyle of the direct and left femur, until the exposure of the subchondral bone of 16 sheep without signs of osteoarticular or systemic alterations. After induction of the lesion, each animal received an implantation site in the lesion of the left pelvic limb and a control treatment (in the lesion of the right pelvic limb (n of 8 per treatment). The control treatments consisted of infiltration of 0.9% NaCl saline solution (Control), or five microfractures in the subchondral bone, followed by infiltration of 0.9% NaCl saline solution (Microfracture). The treatment with test and reference item consisted of fixation of the membranes of the present invention (Test item) and ChondroGide® (Reference item) on the microfracture, using fibrin glue. The animals were exposed to the treatment for 26 weeks. At the end of the period, all animals were anesthetized, euthanized and macroscopically examined. Tissue samples, implant site specimens were processed and sent for imaging and macroscopic analyses.

### 12.2 Analysis Methodology

### 1. Macroscopic Evaluation

The distal segment of the femur, composed of both condyles and femoral trochleas, without the presence of adjacent soft tissues, was photographed and filmed. Of these, 1 video and 3 photos were sent for macroscopic evaluation in a ‘blind trial’, performed by 3 evaluators, 1 veterinarian and 2 physicians, with extensive experience in orthopedic surgeries.

The macroscopic evaluation of the articular cartilage of the medial condyle of the femur was performed, following the guidelines proposed by the International Society of Cartilage Research (ICRS) and classified as Normal, Close to Normal, Abnormal and Severely Abnormal (Table 1).

TABLE
Evaluation of chondral repair according to ICRS.

| | |
|---|---|
| Degree of cartilage repair | |
| At the same level as adjacent normal cartilage | 4 |
| 75% depth fill | 3 |
| 50% depth fill | 2 |
| 25% depth fill | 1 |
| Unrepaired | 0 |
| Integration with the edge of the lesion | |
| Full integration | 4 |
| Integration failure less than 1 mm | 3 |
| ¾ of the integrated fabric, ¼ with | 2 |
| integration failure greater than 1 mm | |
| ½ of the integrated fabric, ½ with | 1 |
| fault greater than 1 mm | |
| Less than ¼ integrated or no integration | 0 |
| Macroscopic appearance | |
| Intact and smooth surface | 4 |
| Fibrillated surface | 3 |
| Few cracks and erosions | 2 |
| Large fissures and erosions | 1 |
| Degeneration of the area | 0 |
| Overall repair assessment | |
| Normal | 12 |
| Close to normal | 11 a 8 |
| Abnormal | 7a4 |
| Severely abnormal/unrepaired | 3 a 1 |

The inter-rater agreement regarding the overall assessment of the repair was analyzed using the Fleiss' kappa coefficient, K (2), since it is a measure used to determine the level of agreement between two or more evaluators when the evaluation method is measured on a categorical scale.

To compare the groups regarding the global assessment of repair, an ordinal multinomial logistic regression model with random effect was proposed. This model allows associating independent variables with a response variable, which is of the ordinal categorical type with more than two levels, also considering the existence of more than one measure for each sample unit, since the same sheep may have been analyzed in knees D and E. The model was still adjusted by side and evaluator.

For all analysis a significance level of 5% was adopted.

### 12.3 Bone Densitometry

Computed tomography images were obtained from the distal segment of the femur, after dissection and removal of adjacent soft tissues through the SHIMADZU equipment, Model: SCT-7800 CT, with 0.5 mm slices, standardized in mA 100 and KVP 120. Computed tomography images were obtained from the 32 knees included in the study. For evaluation, the images were visualized by the Synapse PD-S Viewer software (Fujifilm-Tokyo, Japan), in a transverse plane. The determination of subchondral sclerosis was performed by bone densitometry analysis using the ImageJ software. To this end, 3 images were obtained of each of the knees evaluated in the study (total 96 images) and the area of the condyle selected and classified into pixels. The lateral condyle (negative control) was also evaluated in all images and through the mean of the values was determined basal value of 119,198 pixels.

For comparisons between groups and times regarding bone densitometry variables, the linear regression model with mixed effects (random and fixed effects) adjusted by side was proposed. Linear mixed-effects models are used in data analysis in which responses are grouped (more than one measure for the same individual) and the assumption of independence between observations in the same group is not adequate (3). These models assume that their residuals have a normal distribution with mean 0 and constant variance 02, and have been validated by means of pertinent graphs such as histogram, quantile-quantile and dispersion. For comparisons, orthogonal contrast post-testing was used.

### 12.4 Results

### 1. Global Macroscopic Evaluation

The results of the global macroscopic evaluation are presented in Table below.

TABLE
Results obtained in the global macroscopic evaluation.

| Overall repair assessment | Control | Microfracture |
|---|---|---|
| Nearly normal | 2 (8.33%) | 1 (4.17%) |
| Abnormal | 6 (25%) | 14 (58.33%) |
| Severaly abnormal/no repair | 16 (66.67%) | 9 (37.5%) |
| Overall repair assessment | Test item | Reference item |
| Nearly normal | 10 (41.67%) | 7 (29.17%) |
| Abnormal | 10 (41.67%) | 9 (37.5%) |
| Severaly abnormal/no repair | 4 (16.67%) | 8 (33.33%) |

The results of the analysis of agreement of the evaluators are presented in table below. The results showed good agreement between the evaluators.

TABLE
Analysis of agreement of the evaluators
of the global macroscopic evaluation.

| | | |
|---|---|---|
| Fleiss' kappa | p-value | 95% Confidence interval |

| | | | |
|---|---|---|---|
| 0.183 | 0.205 | 0.174 | 0.192 |

| | Fleiss' | | | |
|---|---|---|---|---|
| Category | kappa | p-value | 95% Confidence interval | |
| Nearly normal | 0.395 | 0.053 | 0.382 | 0.408 |
| Abnormal | −0.067 | 0.744 | −0.079 | −0.054 |
| Severaly | 0.250 | 0.221 | 0.237 | 0.263 |
| abnormal/no repair | | | | |

The results of the comparisons between the different treatments are presented in Table below. There was no significant difference between the macroscopic evaluations of the Microfracture and Control treatments, or of these treatments with the Reference Item. Although there is no statistical difference between the test item and the reference item, unlike the reference item, the test item showed a significant difference when compared to the control and microfracture treatments.

TABLE
Comparisons of global macroscopic evaluation data.

| | Odds | p- | | |
|---|---|---|---|---|
| Comparison | ratio | value | 95% Confidence interval | |
| Microfrature vs Control | 2.52 | 0.12 | 0.80 | 7.98 |
| Test item vs Control | 12.01 | <0.01 | 1.89 | 76.26 |
| Test item 11s Reference item | 2.43 | 0.29 | 0.47 | 12.71 |
| Test item vs Microfrature | 4.77 | 0.03 | 1.21 | 18.73 |
| Reference item vs Control | 4.93 | 0.08 | 0.84 | 28.96 |
| Reference item vs | 1.96 | 0.32 | 0.52 | 7.42 |
| Microfrature | | | | |

### 2. Bone Densitometry

The results of the means of subchondral sclerosis obtained in the evaluation of bone densitometry are presented in Table below.

TABLE
Means of subchondral sclerosis obtained in the evaluation of bone densitometry.

| | Test Item | | Reference Item | | Microfracture | | Control | |
|---|---|---|---|---|---|---|---|---|
| | | Median | | Median | | Median | | Median |
| | Average | (Min; | Average | (Min; | Average | (Min; | Average | (Min; |
| | (SD) | Max | (SD) | Max | (SD) | Max | (SD) | Max |
| Negative | 119.17 | 119.24 | 114.68 | 114.27 | 120.47 | 120.1 | 122.37 | 122.86 |
| Control | (5.04) | (108.27; | (4.95) | (106.66; | (8.05) | (107.38; | (7.54) | (107.79; |
| | | 128.89) | | 124.08) | | 138.71) | | 135.27) |
| Treatment | 134.88 | 135.41 | 136.33 | 135.72 | 140.73 | 143.81 | 140.41 | 138.59 |
| | (8.7) | (116.26; | (6.52) | (125.26; | (10.75) | (123.14; | (9.89) | (123.53; |
| | | 147.81) | | 150.55) | | 154.17) | | 158.18) |

The results of the analyses for comparison of bone densitometry data are presented in Table below. There was a difference between the negative control and all treatments. The test item presented means of subchondral sclerosis lower than the microfracture and control treatments, but did not present statistical difference in relation to the reference item. The reference item presented a lower mean in relation to the control and microfracture treatments, but the difference was not significant. There was no statistical difference between the microfracture and control treatments.

The results demonstrate that the coverage of the osteochondral lesion with the test item was able to reduce the inflammatory process and bone deposition in the subchondral bone (4) and presented results similar to those of the reference item.

TABLE
Comparisons of bone densitometry data.

| | Estimated | p- | | |
|---|---|---|---|---|
| Comparison | difference | value | 95% Confidence interval | |
| Test item (C vs T) | −15.71 | <0.01 | −18.80 | −12.61 |
| Reference item (C vs T) | −21.65 | <0.01 | −24.75 | −18.56 |
| Microfrature (C vs T) | −20.26 | <0.01 | −23.35 | −17.17 |
| Controle (C vs T) | −18.03 | <0.01 | −21.13 | −14.94 |
| T (Test item v, | −1.93 | 0.31 | −5.65 | 1.78 |
| Reference item) | | | | |
| T (Test item vs | −6.56 | 0.04 | −12.81 | −0.32 |
| Microfrature) | | | | |
| T (Test item vs Control) | −6.24 | 0.05 | −12.49 | 0.01 |
| T (Reference item vs | −4.63 | 0.15 | −10.88 | 1.62 |
| Microfrature) | | | | |
| T (Reference item vs | −4.31 | 0.18 | −10.55 | 1.94 |
| Control) | | | | |
| T (Microfrature vs Control) | 0.32 | 0.84 | −2.77 | 3.42 |

### CONCLUSION

From the analyses performed, the present invention presented superiority in relation to the control and microfracture treatments, and equality in relation to the treatment with the reference item.

Further, no adverse reactions were observable at the sheep joint using the present invention. The present invention and collagen scaffold (ChondronGuide) presented superior ICRS scores compared to the control group (P≤0,0001 and P≤0.01, respectively). Cartilage repair in the present invention group was better than in the microfracture-only group (P≤0.01), suggesting that the present invention provides better cell support and promotes chondrogenic differentiation of local mesenchymal cells. In addition, there were no statistical differences between groups in sheep synovial fluid biomarkers.

The results of the study show that the present invention is a safe scaffold that provides better cell support and shows potential improvement in chondrogenic differentiation, which may improve the quality of cartilage repair. In addition, said invention's mechanical and physicochemical properties allow personalization and fixation by suture on the host of them.

Moreover, microfracturing technique in sheep promotes cyst formation, while the present invention has a protective effect and can reduce SCB cyst formation and sclerosis.

### Production Process of the Device of the Present Invention

The process for preparing the device of the present invention comprises the following steps:
a. raw material is placed in a vacuum stove at room temperature;
b. the raw material is weighed to prepare the dissolution with organic solvents;
c. employee performs the machine setup by inserting the appropriate program for the operation;
d. after loading the machine with the polymeric dissolution, the machine is activated, and the production of the membrane takes place with a predetermined flow rate;
e. the membrane blanket is removed and placed in a vacuum stove for residual organic solvent removal;
f. the blanket is sent to the CO2 laser cutter for the membrane cutting process;
g. membranes are stored in an airtight container;
h. the membranes are individually packaged under laminar flow in primary packaging in the form of a polyethylene terephthalate (PET) blister; then the blister is heat-sealed with Tyvek type surgical grade paper; 01 (one) blister of the product is placed in a secondary packaging in the form of a PET/aluminum sachet under laminar flow and the products are placed in an envelope for sterilization in surgical grade paper, whose envelope is thermo-sealed and receives a label indicating sterilization by EtO. This process is carried out to allow the EtO gas to penetrate effectively inside the package, sterilizing the product.

Having described an example of a preferred embodiment of the present invention, it should be understood that the scope of the present invention encompasses other possible variations of the inventive concept described, being limited only by the content of the appended claims, including possible equivalents therein.

1-10. (canceled)

11. A dental or medical device for guided tissue reconstruction and/or regeneration of bone defects and other connective tissue comprising an absorbable synthetic membrane comprised of poly(dioxanone) and having a substantially rectangular cross section having a thickness varying between 0.05 mm and 2.00 mm, a width varying between 15 mm and 200 mm, and a length varying between 20 mm and 200 mm.

12. The device according to claim 11, wherein said device comprises graphene.

13. The device according to claim 12, wherein said device comprises reduced graphene oxide in a concentration that varies from 0.52% to 1.35%.

14. The device according to claim 11 wherein said device comprises stem cells.

15. The device according to claim 14 wherein the stem cells are autogenous adult mesenchymal stem cells obtained from a dermal punch of the patient.

16. The device according to claim 11 wherein said device is configured for a dental application, and wherein said device has a substantially rectangular cross section having a thickness varying between 0.25 mm and 2.00 mm, a width varying between 15 mm and 30 mm, and a length varying between 20 mm and 40 mm.

17. The device according to claim 11 wherein said device is configured for a medical-orthopedic application, and wherein said device has a substantially rectangular cross section having a thickness ranging between 0.25 mm and 2.00 mm, a width varying between 15 mm and 200 mm, and a length varying between 20 mm and 200 mm.

18. The device according to claim 11 wherein the other connective tissue is selected from the group consisting of gingiva, skin, cartilage, and cornea.

19. A method of using the device according to claim 11 comprising using said device for guided tissue reconstruction and/or regeneration of bone defects and other connective tissue.

20. The method according to claim 19 wherein the other connective tissue is selected from the group consisting of gingiva, skin, cartilage, and cornea.

21. The method according to claim 19 wherein said method is used for the treatment of a condition selected from the group consisting of intrabony defects, soft tissue peri-implant defects, and periodontic defects.

22. A process for preparing the dental or medical device according to claim 11, said process comprising:
a. placing raw material in a vacuum greenhouse at room temperature;
b. weighing the raw material to prepare it for dissolution with organic solvents;
c. setting up a machine by inserting an appropriate program to guide operation of the process;
d. loading the machine with polymeric dissolution, activating the machine so as to produce a membrane with a predetermined flow rate, and removing a membrane blanket and placing it into a vacuum greenhouse for residual organic solvent removal;
e. sending the membrane blanket to a CO₂ laser cutter to cur the membrane;
f. storing the membrane in an airtight container; and
g. individually packing the membrane.
